(19) United States
(12) Patent Application Publication
(10) Pub. No.: US 2002/0183631 A1
(43) Pub. Date: Dec. 5, 2002

(54) Method and apparatus for detecting cardiovascular disease

(75) Inventors: Su-Hong Park, Inchon (KR); Dae-Woo Kim, Seoul (KR)

Correspondence Address: COLLIER, SHANNON, SCOTT, PLLC, 3050 K STREET, NW, SUITE 400, WASHINGTON, DC, 20007, US

(73) Assignee: UNIX ELECTRONICS CO., LTD., Inchon (KR)

(21) Appl. No.: 10/140,175

(22) Filed: May 8, 2002

(30) Foreign Application Priority Data
May 8, 2001 (KR) 10-2001-0024889

(51) Int. Cl. A61B 5/02

(52) U.S. Cl. 600/500; 600/501

(57) ABSTRACT

A method is disclosed which comprises the steps of detecting a pulse wave of a user and measuring pulse wave information for a plurality of beats from the pulse wave; producing a statistical average of the measured pulse wave information; and determining whether the user has arrhythmia or high blood pressure based on the produced statistical average of the pulse wave information. And, an apparatus which comprises a detecting part which detects a pulse wave of a user and measures pulse wave information for a plurality of beats from the pulse wave; a memory device which stores the measured pulse wave information; and a determining part which produces a statistical average of the stored pulse wave information and determines whether the user has arrhythmia or high blood pressure based on the produced statistical average of the pulse wave information.

### CROSS REFERENCE TO RELATED PATENT APPLICATION

This application claims priority on Korean Patent Application No. 10-2001-0024889, entitled “Method and Apparatus For Detecting Cardiovascular Disease,” filed May 8, 2001.

1. Field of the Invention

The present invention relates to a method and apparatus for detecting cardiovascular disease, more particularly, to a method and apparatus for detecting arrhythmia or high blood pressure using the statistical average of information taken from pulse wave.

2. Description of the Related Art

Generally, those people who have arrhythmia also have high blood pressure, and they usually have both a sphygmomanometer and a device for measuring electrocardiogram.

The blood pressure is measured with the sphygmomanometer using pulse wave and the electrocardiogram is measured from the electric signal detected from body surface using an electrode attached to the body surface. If the arrhythmia and high blood pressure can be indicated with one apparatus, people will use only one apparatus instead of purchasing two apparatuses.

Information relevant to attempts to address this problem can be found in U.S. Pat. Nos. 5,316,008 and 5,865,761. However, each one of these references exposes one or more of the following disadvantages: those apparatuses are a mere integration of conventional means for measuring blood pressure and electrocardiogram in one apparatus, so they usually result in being bigger than each apparatus individually; also, in measuring the blood pressure or electrocardiogram, abnormal values caused by various reasons which may affect the detection result of the high blood pressure or arrhythmia can occur.

For the foregoing reasons, there is a need for an apparatus and method which can determine both arrhythmia and high blood pressure more conveniently and exclude the effect of abnormal values.

### SUMMARY OF THE INVENTION

The present invention is directed to an apparatus and process that satisfies this need. The method comprises the steps of:

(a) detecting a pulse wave of a user and measuring pulse wave information for a plurality of beats from the pulse wave;

(b) producing a statistical average of the measured pulse wave information; and

(c) determining whether the user has arrhythmia or high blood pressure based on the produced statistical average of the pulse wave information.

The apparatus comprises:

(a) a detecting part which detects a pulse wave of a user and measures pulse wave information for a plurality of beats from the pulse wave;

(b) a memory device which stores the measured pulse wave information; and

(c) a determining part which produces a statistical average of the stored pulse wave information and determines whether the user has arrhythmia or high blood pressure based on the produced statistical average of the pulse wave information.

The present invention provides the method or apparatus, in which high blood pressure and arrhythmia can be detected with one apparatus and, moreover, credible results can be detected using statistical average.

### BRIEF DESCRIPTION OF THE DRAWINGS

These and other features, aspects, and advantages of the present invention will become better understood with regard to the following description, appended claims, and accompanying drawings as follows:

FIG. 1 shows a waveform of an electrocardiogram;

FIG. 2 shows a waveform of a pulse wave;

FIG. 3 shows a schematic block diagram of the apparatus according to one embodiment of the invention;

FIG. 4 shows a flow chart of the method for detecting arrythmia according to one embodiment of the invention; and

FIG. 5 shows a flow chart of the method for detecting high blood pressure according to one embodiment of the invention.

### DETAILED DESCRIPTION OF THE INVENTION

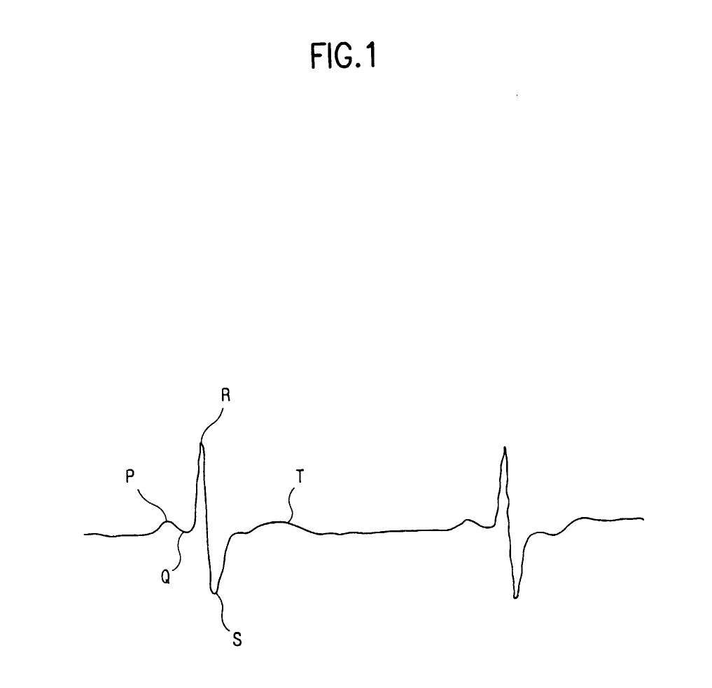

FIG. 1 is a waveform of an electrocardiogram which is used to explain the concept of the present invention. As shown in FIG. 1, the electrocardiogram has P, Q, R, S, T part in a time period.

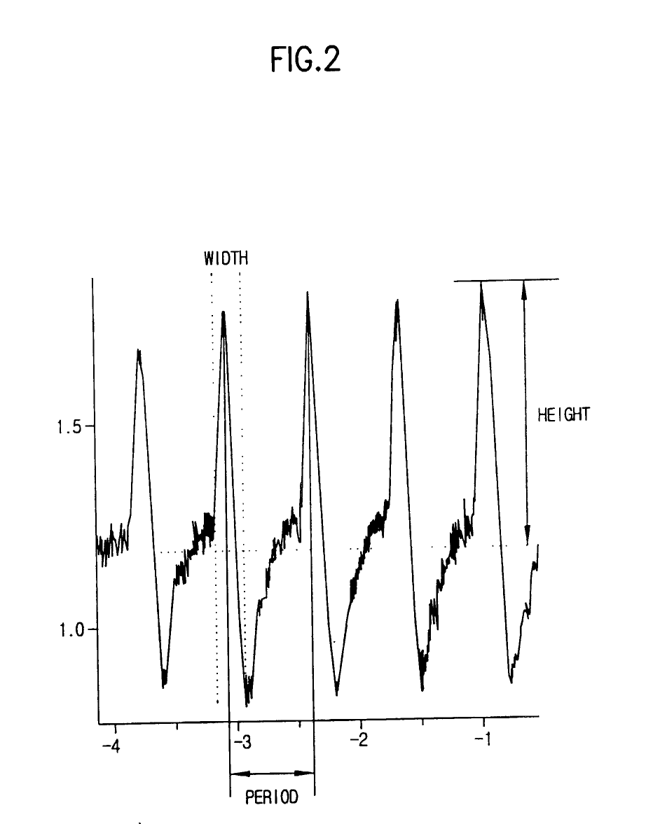

FIG. 2 is a waveform of a pulse wave. Pulse wave information such as a width, height and time period, which is used in the present invention is shown. The pulse wave and the electrocardiogram have a certain relationship with each other. That is the period of the pulse wave is the same as the interval between the consecutive R parts of the electrocardiogram. So, the arrhythmia that is detected with the electrocardiogram also can be detected with the pulse wave.

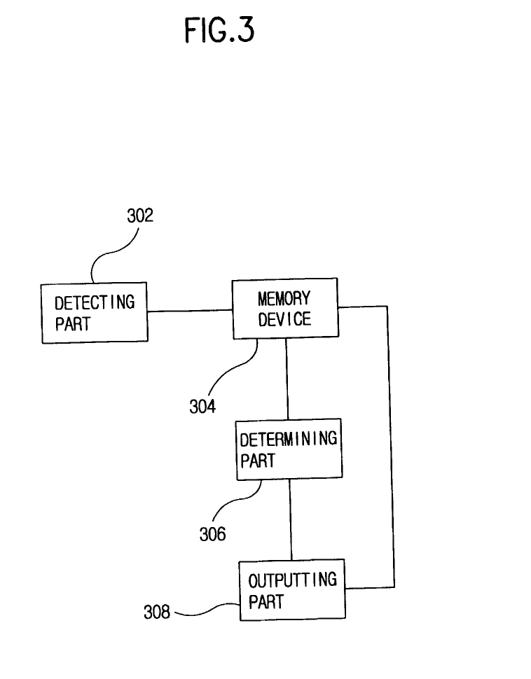

FIG. 3 shows a schematic block diagram of the apparatus according to one embodiment of the invention.

First, the situation for detecting arrhythmia is described. A detecting part 302 detects a user's pulse wave and measures pulse wave information for a plurality of beats from the pulse wave. The pulse wave information comprises at least one kind of information among pulse wave information such as a period, width and height of the pulse wave.

A memory device 304 stores the measured pulse wave information sent from detecting part 302.

A determining part 306 sorts each kind of pulse wave information according to size. And the determining part 306 sets the average of two central pieces of the sorted pulse wave information as a statistical average of the pulse wave information when the total number of the detected beats is even, whereas the determining part 306 sets the centermost piece of the pulse wave information as the statistical average when the total number of the beats is odd.

The determining part 306 compares the pulse wave information for each beat with the statistical average of the pulse wave information respectively, and concludes that the user has arrhythmia when the total number of the beats of which the pulse wave information is outside of a predetermined range from the statistical average of the pulse wave information is greater than a predetermined value.

The determining part 306 concludes that the user is in normal state when the total number of the beats of which the pulse wave information is within the predetermined range from the statistical average of the pulse wave information is smaller than the predetermined value.

An outputting part 308 outputs either the conclusion or the statistical average of the pulse wave information or both of them.

Next, the situation for detecting high blood pressure is described. The detecting part 302 detects a user's pulse wave and measures a plurality of blood pressure values from the pulse wave.

The memory device 304 stores the measured blood pressure values, a predetermined reference value of the blood pressure values.

First, the determining part 306 sorts the blood pressure values according to their sizes.

Next, the determining part 306 sets the average of the two central blood pressure values of the sorted blood pressure values as a statistical average of the blood pressure values when the total number of the blood pressure values is even, and sets the centermost blood pressure value as a statistical average of the blood pressure values when the total number of the high blood pressure values is odd.

Finally, the determining part 306 concludes that the user has high blood pressure when the statistical average of the blood pressure values is greater than the reference value of the blood pressure values, whereas the user is in normal state when the statistical average of the blood pressure values is smaller than the reference value of the blood pressure values.

The outputting part 308 outputs either the conclusion or the statistical average of the blood pressure values or both of them.

In the above, the apparatus is used for detecting the arrhythmia and high blood pressure respectively, but the apparatus can be used to perform both operations simultaneously.

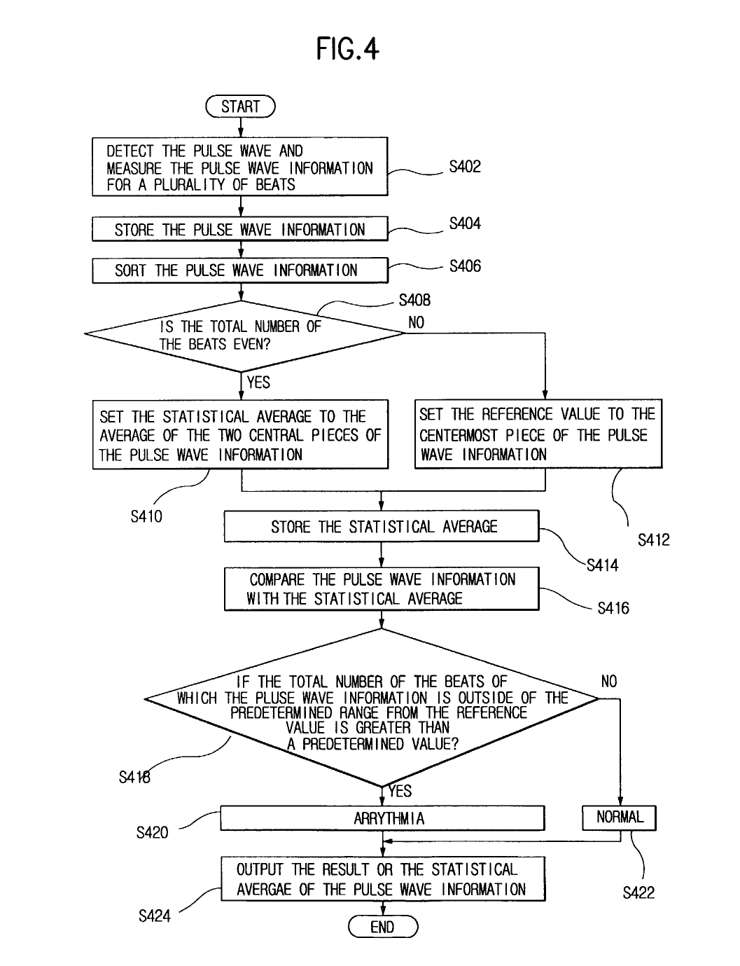

FIG. 4 shows a flow chart of the method for detecting arrhythmia according to one embodiment of the invention. The detecting part 302 detects a user's pulse wave and measures pulse wave information for a plurality of beats from the pulse wave S402.

The pulse wave information comprises at least one kind of information among a period, width and height. The plural number of the beats is necessary to prevent the determination of arrhythmia with only a single detection value. The required number of the beats varies according to required reliability of the conclusion.

The memory device 304 stores the pulse wave information sent from detecting part S404.

The determining part 306 sorts the pulse wave information stored in the memory device 304 according to size S406, and checks whether the total number of the beats for each kind of the pulse wave information is even or odd S408.

The statistical average of the pulse wave information is an average of two central pieces of the pulse wave information when the total number of the beats is even S410. For example, if the total number of the detected beats for a kind of pulse wave information is 4, the statistical average is the average of the second and third pieces of the pulse wave information among the sorted pulse wave information.

On the other hand, the statistical average is the centermost piece of the sorted pulse wave information when the total number of the beats is odd S412. For example, if the total number of the detected beats for a kind of the pulse wave information is 5, the statistical average is the third piece of the pulse wave information among the sorted pulse wave information.

The memory device 304 stores the statistical average of each kind of the pulse wave information S414. The use of the statistical average can exclude the effect of a few abnormal values caused by various reasons from the detection result of arrhythmia.

The determining part 306 compares the pulse wave information for each beat with the statistical average of the pulse wave information respectively S416.

And the determining part 306 checks whether the total number of the beats of which the pulse wave information is outside of a predetermined range from the statistical average is greater than a predetermined value S418.

The determining part 306 concludes that the user has arrhythmia when the number is greater than the predetermined value S420, otherwise the determining part 306 concludes that the user is in normal state S422.

The predetermined range and the predetermined value can be selected, and preferably have the value of 20% and 2 respectively. So, if the pulse wave information is smaller than 80% or greater than 120% of the statistical average of the pulse wave information more than 2 times, the determining part 306 concludes that arrhythmia exists.

Finally, the outputting part 308 outputs either the conclusion or the statistical average of the pulse wave information or both of them according to a predetermined setting S424.

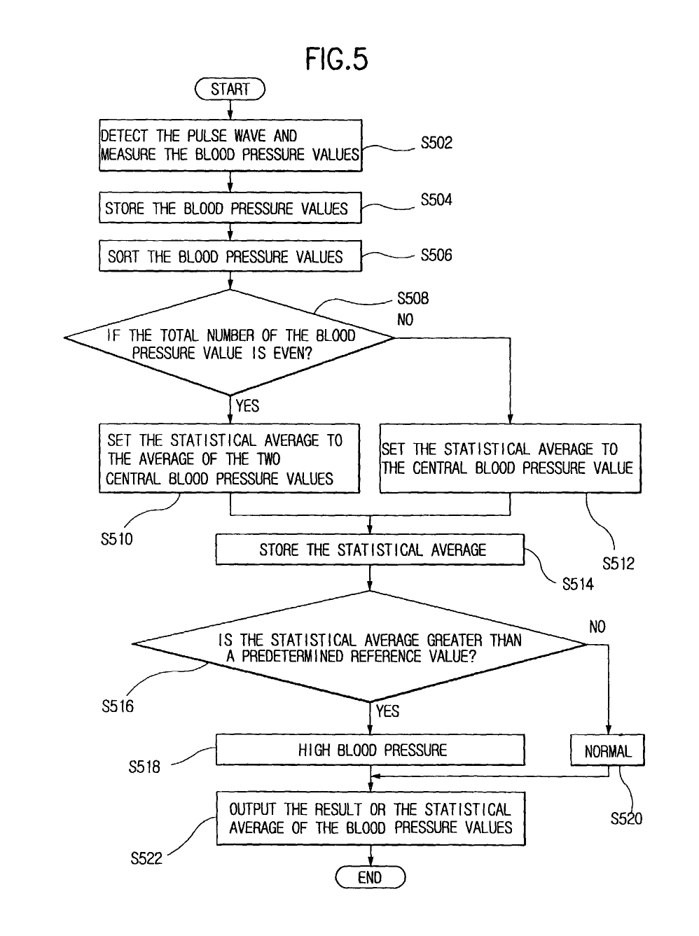

FIG. 5 shows a flow chart of the method for detecting high blood pressure according to one embodiment of the invention.

The detecting part 302 detects a user's pulse wave and measures a plurality of blood pressure values from the pulse wave S502. The memory device 304 stores the blood pressure values S504.

The determining part 306 sorts the blood pressure values according to their sizes S506 and checks whether the total number of the blood pressure values is even or odd S508.

The statistical average of the blood pressure values is the average of the two central blood pressure values of the sorted blood pressure values when the total number of detected blood pressure values is even S510, whereas the statistical average of the blood pressure values is the centermost blood pressure value when the total number of the blood pressure values is odd S512.

The memory device 304 stores the statistical average of the blood pressure values S514.

The determining part 306 compares the statistical average of the blood pressure values with a predetermined blood pressure reference value S516.

The determining part 306 concludes the user has high blood pressure when the statistical average of the blood pressure value is greater than the blood pressure reference value S518.

The blood pressure reference value can be selected and preferably has the value of systolic blood pressure 140mmHg or diastolic blood pressure 90mmHg.

So, the determining part 306 concludes that the user has high blood pressure when the systolic blood pressure is higher than 140mmHg or the diastolic blood pressure is higher than 90mmHg.

The determining part 306 concludes that the user doesn't have high blood pressure when both systolic and diastolic blood pressure are lower than each reference value of the blood pressure S520.

For example, if the systolic blood pressure is lower than 140mmHg and the diastolic blood pressure is lower than 90mmHg, the determining part 306 concludes that the user does not have high blood pressure.

If the results from the systolic blood pressure and the diastolic blood pressure are different, the determining part 306 determines that high blood pressure exists.

Finally, the outputting part 308 outputs either the conclusion or the statistical average of the blood pressure values, or both of them according to a predetermined setting S522.

The previously described versions of the present invention have many advantages. Although the present invention has been described in considerable detail with reference to certain preferred versions there of, other versions are possible. Therefore, the sprit and scope of the appended claims should not be limited to the description of the preferred versions contained herein.

What is claimed is:

1. A method for detecting cardiovascular disease comprising the steps of:
(a) detecting a pulse wave of a user and measuring pulse wave information for a plurality of beats from the pulse wave;
(b) producing a statistical average of the measured pulse wave information; and
(c) determining whether the user has arrhythmia or high blood pressure based on the produced statistical average of the pulse wave information.

2. The method of claim 1, the step (b) comprises the steps of:
sorting the measured pulse wave information according to size; and
setting the average of two central pieces of the sorted pulse wave information as the statistical average when the total number of the beats is even and setting the centermost piece of the sorted pulse wave information as the statistical average when the total number of the beats is odd.

3. The method of claim 1, wherein the pulse wave information comprises at least one kind of information among a period, width, or height of the pulse wave.

4. The method of claim 3, wherein the step of determining arrhythmia comprises the steps of:
comparing the pulse wave information for each beat with the statistical average of the pulse wave information; and
concluding that the user has arrhythmia when the total number of the beats of which the pulse wave information is outside of a predetermined range from the statistical average of the pulse wave information is greater than a predetermined value.

5. The method of claim 1, wherein the pulse wave information comprises blood pressure values of the user.

6. The method of claim 5, wherein the step of determining high blood pressure comprises the steps of:
comparing the statistical average of the pulse wave information with a predetermined blood pressure reference value; and
concluding that the user has high blood pressure when the statistical average of the pulse wave information is greater than the predetermined blood pressure reference value.

7. The method of claim 1, further comprising the step of outputting the determination result.

8. The method of claim 1 or 7, further comprising the step of outputting the statistical average of the pulse wave information

9. Apparatus for detecting cardiovascular disease comprising:
(a) a detecting part which detects a pulse wave of a user and measures pulse wave information for a plurality of beats from the pulse wave;
(b) a memory device which stores the measured pulse wave information; and
(c) a determining part which produces a statistical average of the stored pulse wave information and determines whether the user has arrhythmia or high blood pressure based on the produced statistical average of the pulse wave information.

10. The apparatus of claim 9, wherein the determining part produces the statistical average of the pulse wave information by sorting the pulse wave information according to size, and then setting the average of two central pieces of the stored pulse wave information as the statistical average when the total number of the beats is even and setting the centermost piece of the stored pulse wave information as the statistical average when the total number of the beats is odd.

11. The apparatus of claim 9, wherein the pulse wave information comprises at least one kind of information among a period, width, and height of the pulse wave.

12. The apparatus of claim 11, wherein the determining part compares the pulse wave information for each beat with the statistical average of the pulse wave information respectively and concludes that the user has arrhythmia when the total number of the beats of which the pulse wave information is outside of a predetermined range from the statistical average of the pulse wave information is greater than a predetermined value.

13. The apparatus of claim 9, wherein the pulse wave information comprises the blood pressure values of the user.

14. The apparatus of claim 13, wherein the determining part compares the statistical average of the pulse wave information with a predetermined blood pressure value and concludes that the user has high blood pressure when the statistical average of the pulse wave information is greater than the predetermined blood pressure reference value.

15. The apparatus of claim 9, further comprising an outputting part which outputs the determination result or the statistical average of the pulse wave information.
